(19) United States
(12) Patent Application Publication
(10) Pub. No.: US 2023/0141434 A1
(43) Pub. Date: May 11, 2023

(54) DIRECT IN VIVO REPROGRAMMING USING TRANSCRIPTION FACTOR ETV2 GENE FOR ENDOTHELIAL CELL AND VESSEL FORMATION

(71) Applicant: EMORY UNIVERSITY, Atlanta, GA (US)

(72) Inventors: Young-Sup YOON, Atlanta, GA (US); Sang Ho Lee, Atlanta, GA (US); Seongho BAE, Atlanta, GA (US)

(21) Appl. No.: 18/000,865

(22) PCT Filed: Jun. 14, 2021
(86) PCT No.: PCT/US2021/037261

§ 371 Date: Dec. 6, 2022

Related U.S. Application Data
(60) Provisional application No. 63/038,215, filed on Jun. 12, 2020.

(51) Int. Cl. A61K 35/34; C12N 15/86; A61K 38/17; A61K 9/00; A61P 9/10; C12N 5/071

(52) U.S. Cl. CPC A61K 35/34; C12N 15/86; A61K 38/1709; A61K 9/0019; A61P 9/10; C12N 5/069; C12N 2506/1307

(57) ABSTRACT

This disclosure relates to using a ETV2 gene or gene products including DNA, RNA, mRNA, ETV2 proteins, or protein containing exosomes, to directly reprogram and convert resident non-endothelial cells of host into endothelial cells in situ, i.e., in places of the body or tissue where ETV2 is injected. In certain embodiments, it is contemplated that directly reprogrammed and converted endothelial cells will enhance blood vessel regeneration in the tissues where blood vessels have been damaged.

### CROSS-REFERENCE TO RELATED APPLICATIONS

This application claims the benefit of U.S. Provisional Application No. 63/038,215 filed Jun. 12, 2020. The entirety of this application is hereby incorporated by reference for all purposes.

### STATEMENT REGARDING FEDERALLY SPONSORED RESEARCH OR DEVELOPMENT

This invention was made with government support under HL127759 and HL129511 awarded by the National Institutes of Health. The government has certain rights in the invention.

### BACKGROUND

Ischemic cardiovascular diseases, which include coronary artery disease (e.g. myocardial infarction) and peripheral artery disease (e.g. critical limb ischemia), are a frequent cause of morbidity and mortality. The main cause of these clinical entities is the loss of blood vessels. Endothelial cells (ECs), as a key element of vasculature, are indispensable for repairing injured or ischemic tissues. Thus, there is a need to identify improved methods of therapeutically managing these diseases and related conditions.
Margariti et al. report reprogramming of fibroblasts into endothelial cells capable of angiogenesis and re-endothelialization in tissue-engineered vessels. Proc Natl Acad Sci USA, 2012, 109:13793-13798.
Lee et al. report direct reprogramming of human dermal fibroblasts into endothelial cells using ETV2. Circulation, 2014, 130: A18205.
Morita et al. report ETV2 directly converts human fibroblasts into functional endothelial cells. Proc Natl Acad Sci USA, 2015, 112(1):160-165.
Liu et al. report induction of hematopoietic and endothelial cell program orchestrated by ETS transcription factor ER71/ETV2. EMBO reports (2015) embr.201439939.
Lee et al., report direct reprogramming of human dermal fibroblasts into endothelial cells using ER71/ETV2. Circulation Research. 2017, 120:848-861.
Lee et al., report vascular regeneration with new sources of endothelial cells. Circ Res. 2019, 124(1): 29-31.
Lee et al., report in vivo transduction of ETV2 improves cardiac function and induces vascular regeneration following myocardial infarction. Experimental & Molecular Medicine (2019) 51:13.
Yoon et al. report endothelial or endothelial like cells cultured from fibroblasts exposed to transcription factor ETV2. U.S. Pat. No. 10,023,842.

References cited herein are not an admission of prior art.

### SUMMARY

This disclosure relates to using a ETV2 gene or gene products including DNA, RNA, mRNA, ETV2 proteins, or protein containing exosomes, to directly reprogram and convert resident non-endothelial cells of host into endothelial cells in situ, i.e., in places of the body or tissue where the ETV2 material is injected. In certain embodiments, it is contemplated that directly reprogrammed and converted endothelial cells will enhance blood vessel regeneration in the tissues where blood vessels have been damaged and new blood vessels are demanded for proper function.

In certain embodiments, direct delivery of ETV2 material can be used to treat diseases requiring revascularization including, but not limited to, coronary artery diseases, myocardial infarction, heart failure, peripheral artery diseases, critical limb ischemia, stroke, diabetic complications, and would healing. In certain embodiments, the ETV2 gene or gene products can be delivered to the sites of diseases by local injection in various forms such as in lentivirus, retrovirus, adenovirus, adeno-associated virus (AAV), and mRNA.

In certain embodiments, this disclosure relates to methods of converting non-endothelial cells into endothelial cells. In certain embodiments, the non-endothelial cells that convert into endothelial cells are within a diameter of 10, 20, 30, 40, 50, 60, 70, 80, 90, or 100 mm from an injection site. In certain embodiments, the tissue is muscular tissue or heart tissue.

In certain embodiments, this disclosure relates to methods of converting non-endothelial cells into endothelial cells comprising administering ETV2 materials such as proteins, exosomes, nucleic acids or vector encoding transcription factor ETV2 as reported herein, to a subject in places of the body or tissue wherein non-endothelial cells at an injection site or in the vasculature convert into endothelial cells.

In certain embodiments, the nucleic acid is DNA or RNA encoding ETV2. In certain embodiments, a nucleic acid or vector encoding ETV2 is a recombinant lentivirus, retrovirus, adenovirus, or adeno-associated virus (AAV). In certain embodiments, the nucleic acid is mRNA encoding ETV2. In certain embodiments, the ETV2 protein is contained within or on the outside surface of an exosome or other particulate structure.

In certain embodiments, this disclosure relates to methods of generating a blood vessel in a tissue comprising administering ETV2 materials, e.g., ETV2, nucleic acids, proteins, conjugates, or particles as described herein, to a subject in a tissue wherein non-endothelial cells at the injection site convert into endothelial cells or blood vessels, or endothelial cells at the injection site convert into blood vessels.

In certain embodiments, this disclosure relates to methods of enhancing blood vessel regeneration in tissues where blood vessels have been damaged comprising administering ETV2 materials to a subject in a tissue wherein non-endothelial cells at the injection site convert into endothelial cells or blood vessels, or endothelial cells at the injection site convert into blood vessels.

In certain embodiments, this disclosure relates to methods of producing new blood vessels in tissues comprising administering ETV2 materials to a subject in a tissue wherein non-endothelial cells or endothelial cells at the injection site convert into blood vessels.

In certain embodiments, this disclosure relates to methods of treating disease requiring revascularization comprising administering ETV2 materials to a subject in a tissue wherein non-endothelial cells or endothelial cells at the injection site convert into endothelial cells and/or blood vessels.

In certain embodiments, the disease requiring revascularization is coronary artery diseases, myocardial infarction, heart failure, peripheral artery diseases, critical limb ischemia, stroke, diabetic complications, and would healing. In certain embodiments, this disclosure relates to methods of producing or treating disease requiring revascularization comprising administering ETV2 materials to a subject in a tissue wherein non-endothelial cells or endothelial cells at the injection site are converted or develop into endothelial cells and/or blood vessels. In certain embodiments, the disease requiring revascularization is coronary artery diseases, myocardial infarction, heart failure, peripheral artery diseases, critical limb ischemia, stroke, diabetic complications, and would healing. Other contemplated diseases for treatment include angina, calcific aortic valve disease, hypertensive heart disease, rheumatic heart disease, cardiomyopathy, heart arrhythmia, congenital heart disease, valvular heart disease, carditis, aortic aneurysms, thromboembolic disease, and venous thrombosis.

### BRIEF DESCRIPTION OF THE SEVERAL VIEWS OF THE DRAWINGS

FIG. 1 shows confocal microscopic images of Ad-ETV2 injected ischemic hindlimb tissue (right) and heart tissue with myocardial infarction (left) of Fsp1-Cre;R26R-tdTomato mouse. Fibroblasts were labeled with tdTomato whose expression was induced by Fsp1 promoter driven Cre recombinase, whereas perfusable blood vessels were labeled with BSL1. Arrowhead: un-incorporated or un-reprogrammed or free fibroblasts (FSP1+), Arrow: incorporated and reprogrammed fibroblasts, Scale bar: 50 μm.

FIG. 2A shows data from ejection fraction of Ad-ETV2 injected heart with myocardial infarction. Echocardiography was performed at one, three and four weeks after induction of myocardial infarction with (ETV2) or without (control) Ad-ETV2 injection (n=3). Ejection fraction of each time points were compared between Ad-ETV2 treated and non-treated group.

FIG. 2B shows fold changes in ejection fraction of third and fourth weeks compared to the first week.

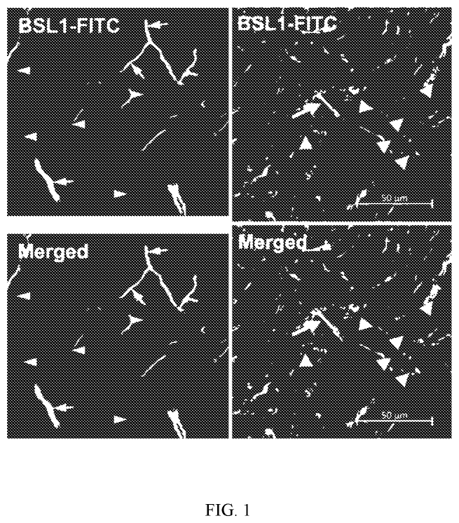

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

Use of the term “embodiments” infers that such element(s) are example(s), but not necessarily limited to the example(s).

It must be noted that, as used in the specification and the appended claims, the singular forms “a,” “an,” and “the” include plural referents unless the context clearly dictates otherwise. Thus, for example, reference to “a nucleic acid” or “a vector” includes reference to one or more nucleic acids or vectors and equivalents thereof known to those skilled in the art, and so forth. It is further noted that the claims may be drafted to exclude any optional element.

Unless the context requires otherwise, throughout the specification and claims which follow, the word “comprise” and variations thereof, such as, “comprises,” “comprising” “including,” “containing,” or “characterized by,” are to be construed in an open, inclusive sense, that is, as “including, but not limited to” and does not exclude additional, unrecited elements or method steps. By contrast, the transitional phrase “consisting of” excludes any element, step, or ingredient not specified in the claim that do not materially affect the basic and novel characteristic(s) of the claimed invention. In embodiments or claims where the term “comprising” is used as the transition phrase, such embodiments can also be envisioned with replacement of the term “comprising” with the terms “consisting of.”

The term “ETV2” refers to transcription factor ets variant 2 (ETV2). Human ETV2 has three isoforms as reported in NCBI Reference Sequence: NP_055024.2, NP_001287903.1, and NP_001291478.1. All isoforms are contemplated for uses disclosed herein. Variant ETV2 proteins can be produced by mutating a vector to produce appropriate codon alternatives for polypeptide translation. Active variants and fragments can be identified with a high probability using computer modeling. Shihab et al. report an online genome tolerance browser. BMC Bioinformatics, 2017, 18(1):20. Ng et al. report methods of predicting the effects of amino acid substitutions on protein function. Annu Rev Genomics Hum Genet, 2006, 7:61-80. Teng et al. Approaches and resources for prediction of the effects of non-synonymous single nucleotide polymorphism on protein function and interactions. Curr Pharm Biotechnol, 2008, 9(2):123-33.

Endothelial cells form the tissue in contact with the blood stream, e.g., as the lining of blood vessels, capillaries, and the heart. Vascular endothelial growth factor receptor 2 (VEGFR2, product of the gene KDR) is expressed on certain endothelial cells and endothelial progenitor cells. Cadherin 5, type 2 (VE-Cadherin, product of the gene CDH5) in expressed on the vascular endothelium. See also Müller et al., Expression of the endothelial marker platelet/endothelial cell adhesion molecule 1 (PECAM-1), Von Willebrand factor (vWF), and CD34 in vivo and in vitro. Exp Mol Pathol. 2002, 72(3):221-9. The endothelial tyrosine kinase receptor, Tie2 also known as TEK, is a marker of the endothelial phenotype. Anghelina et al. J Cell Mol Med. 2005, 9(1):113-21.

A skilled artisan would understand that one could produce a large number of operable variants that would be expected to have the desirable binding properties. ETV2 genes are known and members shared significant homologies from one species to another. The sequences are not identical. Some are conserved substitutions (plus sign). Some are not conserved substitutions. A skilled artisan is able to identify numerous variant operable embodiments. Skilled artisans would not blindly try random combinations, but instead utilize computer programs to make stable substitutions. Active variants and fragments can be identified with a high probability using computer modeling. Skilled artisans would know that certain conserved substations would be desirable. In addition, a skilled artisan would not typically alter evolutionary conserved positions. See Saldano et al. Evolutionary Conserved Positions Define Protein Conformational Diversity, PLoS Comput Biol. 2016, 12(3): e1004775.

Desired amino acid substitutions (whether conservative or non-conservative) can be determined by those skilled in the art at the time such substitutions are desired. Guidance in determining which and how many amino acid residues may be substituted, inserted or deleted without abolishing biological activity may be found using computer programs well known in the art, for example, RaptorX, ESyPred3D, HHpred, Homology Modeling Professional for HyperChem, DNAStar, SPARKS-X, EVfold, Phyre, and Phyre2 software. See also Saldano et al. Evolutionary Conserved Positions Define Protein Conformational Diversity, PLoS Comput Biol. 2016, 12(3): e1004775; Marks et al. Protein structure from sequence variation, Nat Biotechnol. 2012, 30(11):1072-80; Mackenzie et al. Curr Opin Struct Biol, 2017, 44:161-167 Mackenzie et al. Proc Natl Acad Sci USA. 113(47): E7438-E7447 (2016); Joseph et al. J R Soc Interface, 2014, 11(95):20131147, Wei et al. Int. J. Mol. Sci. 2016, 17(12), 2118. Variants can be tested in functional assays. Certain variants have less than 10%, and preferably less than 5%, and still more preferably less than 2% changes (whether substitutions, deletions, and so on).

In certain embodiments, this disclosure contemplates that ETV2 proteins disclosed herein may be variants. Variants may include 1 or 2 amino acid substitutions or conserved substitutions. Variants may include 3 or 4 amino acid substitutions or conserved substitutions. Variants may include 5 or 6 or more amino acid substitutions or conserved substitutions. Variant include those with not more than 1% or 2% of the amino acids are substituted. Variant include those with not more than 3% or 4% of the amino acids are substituted. Variants include proteins with greater than 80%, 89%, 90%, 95%, 98%, or 99% identity or similarity.

Sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q. The amino acid groups are also considered conserved substitutions.

Percent identity can be determined, for example, by comparing sequence information using the GAP computer program, version 6.0, available from the University of Wisconsin Genetics Computer Group (UWGCG). The GAP program utilizes the alignment method of Needleman and Wunsch (J Mol Biol 1970 48:443), as revised by Smith and Waterman (Adv Appl Math 1981 2:482). Briefly, the GAP program defines identity as the number of aligned symbols (i.e., nucleotides or amino acids) which are identical, divided by the total number of symbols in the shorter of the two sequences. The preferred default parameters for the GAP program include: (1) a unitary comparison matrix (containing a value of 1 for identities and 0 for non-identities) and the weighted comparison matrix of Gribskov and Burgess (Nucl Acids Res 1986 14:6745), as described by Schwartz and Dayhoff (eds., Atlas of Protein Sequence and Structure, National Biomedical Research Foundation, Washington, D.C. 1979, pp. 353-358); (2) a penalty of 3.0 for each gap and an additional 0.10 penalty for each symbol in each gap; and (3) no penalty for end gaps.

“Subject” means any animal, but is preferably a mammal, such as, for example, a human, monkey, mouse, or rabbit.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

The term “effective amount” refers to that amount of a compound, peptide, nucleic acid, vector, or pharmaceutical composition described herein that is sufficient to effect the intended application including, but not limited to, disease treatment. In relation to a combination therapy, an “effective amount” indicates the combination of agent results in synergistic or additive effect when compared to the agents individually. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (mRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage, and polyadenylation.

A “heterologous” nucleic acid sequence or peptide sequence refers to a nucleic acid sequence or peptide sequence that do not naturally occur, e.g., because the whole sequences contain a segment from other plants, bacteria, viruses, other organisms, or joinder of two sequences that occur the same organism but are joined together in a manner that does not naturally occur in the same organism or any natural state.

The term “heterologous gene” refers to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript).

The term “a polynucleotide having a nucleotide sequence encoding a gene” or “a nucleic acid sequence encoding” a specified polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. In contrast, a “regulatable” or “inducible” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occurring in nature. The segments may be joined in an altered arrangement such that the entire nucleic acid sequence from start to finish does not naturally occur. The term “recombinant” when made in reference to a protein or a polypeptide refers to a protein molecule that is expressed using a recombinant nucleic acid molecule.

In certain embodiments, this disclosure contemplates a recombinant vector encoding ETV2. In certain embodiments, the recombinant vector is a plasmid or viral vector, lentiviral vector, adenoviral vector, or adeno-associated virus (AAV) vector. Viral vectors encapsidate genomes wherein most protein-coding sequences have been removed and have therapeutic gene expression cassettes designed in their place, e.g., to express ETV2. The sequences of viral origin are typically the viral inverted terminal repeats (ITRs), which guide genome replication and packaging during vector production.

The term “recombinant vector” when made in reference to vectors and nucleic acids refers to a nucleic acid molecule that is comprised of segments of nucleic acid joined together by means of molecular biological techniques. The term recombinant nucleic acid is distinguished from the natural recombinants that result from crossing-over between homologous chromosomes. Recombinant nucleic acids as used herein are an unnatural union of nucleic acids from nonhomologous sources, usually from different organisms. Recombinant vectors can comprise any type of nucleotides, including, but not limited to DNA and RNA, which can be single-stranded or double-stranded, synthesized or obtained in part from natural sources, and which can contain natural, non-natural, or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector. The recombinant vector of the invention can be any suitable recombinant vector and can be used to transform or transfect any suitable host cell. Suitable vectors include those designed for propagation and expansion or for expression or both, such as plasmids and viruses.

In certain embodiments, the recombinant vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like.

In certain embodiments, the vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), FLAG tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

A “selectable marker” is a nucleic acid introduced into a vector that encodes a polypeptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme which confers a blue color in the presence of X-gal (5-bromo-4-chloro-3-indolyl-β-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: AmpR, CamR, TetR, BlasticidinR, NeoR, HygR, AbxR, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (at1D), UDP-glucose:galactose-1-phosphate uridyltransferasel (galT), feedback-insensitive α subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dh1A), mannose-6-phosphate reductase gene (M6PR), hygromycin phosphotransferase (HPT), and D-serine ammonialyase (dsdA).

The recombinant vector can comprise a native or nonnative promoter operably linked to the nucleotide sequence encoding the ETV2 protein (including functional variants thereof), or to the nucleotide sequence which is complementary to or which hybridizes to the nucleotide sequence encoding the ETV2 protein (including functional variants thereof). The selection of promoters, e.g., strong, weak, inducible, tissue-specific, and developmental-specific, is within the ordinary skill of the artisan. Similarly, the combining of a nucleotide sequence with a promoter is also within the skill of the artisan.

The recombinant vectors can be designed for either transient expression, for stable expression, or for both. Also, the recombinant vectors can be made for constitutive expression or for inducible expression. Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine deaminase, purine nucleoside phosphorylase, and nitroreductase.

Protein “expression systems” refer to in vivo and in vitro (cell free) systems. Systems for recombinant protein expression typically utilize somatic cells transfected with a DNA expression vector that contains the template. The cells are cultured under conditions such that they translate the desired protein. Expressed proteins are extracted for subsequent purification. In vivo protein expression systems using prokaryotic and eukaryotic cells are well known. Also, some proteins are recovered using denaturants and protein-refolding procedures. In vitro (cell-free) protein expression systems typically use translation-compatible extracts of whole cells or compositions that contain components sufficient for transcription, translation and optionally post-translational modifications such as RNA polymerase, regulatory protein factors, transcription factors, ribosomes, tRNA cofactors, amino acids and nucleotides. In the presence of an expression vector, these extracts and components can synthesize proteins of interest. Cell-free systems typically do not contain proteases and enable labelling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

In certain embodiments, this disclosure relates to a host cell comprising any of the recombinant expression vectors described herein. As used herein, the term “host cell” refers to any type of cell that can contain the recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5alpha E. coli cells, Chinese hamster ovarian cells, monkey VERO cells, COS cells, HEK293 cells, and the like.

### Methods of Use

This disclosure relates to using ETV2 materials, e.g., gene or gene products including mRNA, proteins, protein containing exosomes, to directly reprogram and convert resident non-endothelial cells of host into endothelial cells or vessels in situ, i.e., in places of the body or tissue where ETV2 material is injected. In certain embodiments, it is contemplated that directly reprogrammed and converted endothelial cells will enhance blood vessel regeneration in the tissues where blood vessels have been damaged and new blood vessels are demanded for proper function of body. In certain embodiments, direct delivery of ETV2 materials can be used to treat diseases requiring revascularization including, but not limited to, coronary artery diseases, myocardial infarction, heart failure, peripheral artery diseases, critical limb ischemia, stroke, diabetic complications, and would healing. In certain embodiments ETV2 materials can be delivered to the sites of diseases by a local injection site in various forms such as in lentivirus, retrovirus, adenovirus, adeno-associated virus (AAV), and mRNA.

In certain embodiments, this disclosure relates to methods of converting non-endothelial cells into endothelial cells comprising administering a nucleic acid or vector encoding transcription factor ETV2 to a subject in places of the body or tissue wherein non-endothelial cells at the injection site or in the vasculature convert into endothelial cells or vessels.

In certain embodiments, the injection site is muscle tissue or cardiac muscle tissue. In certain embodiments, the muscle tissue is striated muscle or skeletal muscle. In certain embodiments, the cardiac muscle tissue is the myocardium, pericardium, or endocardium.

In certain embodiments, this disclosure relates to methods of converting non-endothelial cells into endothelial cells or vessels. In certain embodiments, the non-endothelial cells that convert into endothelial cells or vessels are within a diameter of 10, 20, 30, 40, 50, 60, 70, 80, 90, or 100 mm from an injection site. In certain embodiments, the tissue is muscular tissue or heart tissue.

In certain embodiments, the nucleic acid is mRNA encoding ETV2. In certain embodiments, the nucleic acid is DNA or RNA encoding ETV2. In certain embodiments, nucleic acid or vector encoding ETV2 is a recombinant lentivirus, retrovirus, adenovirus, or adeno-associated virus (AAV).

In certain embodiments, this disclosure relates to methods of converting non-endothelial cells into endothelial cells comprising administering an ETV2 protein or functional fragment thereof to a subject in places of the body or tissue wherein non-endothelial cells at the injection site or in the vasculature convert into endothelial cells or vessels. In certain embodiments, this disclosure relates to methods wherein the protein is contained within an exosome or other particulate structure.

In certain embodiments, this disclosure relates to methods of generating blood vessels in a tissue comprising administering a nucleic acid or vector encoding transcription factor ETV2 to a subject in a tissue wherein non-endothelial cells at the injection site convert into endothelial cells or vessels.

In certain embodiments, the nucleic acid is mRNA encoding ETV2. In certain embodiments, the nucleic acid is DNA or RNA encoding ETV2. In certain embodiments, nucleic acid or vector encoding ETV2 is a recombinant lentivirus, retrovirus, adenovirus, or adeno-associated virus (AAV).

In certain embodiments, this disclosure relates to methods of generating blood vessels in a tissue comprising administering an ETV2 protein or functional fragment thereof to a subject in a tissue wherein non-endothelial cells at the injection site convert into endothelial cells or vessels. In certain embodiments, the protein is contained within an exosome or other particulate structure.

In certain embodiments, this disclosure relates to methods of generating blood vessels in a tissue. In certain embodiments, the blood vessels are generated are within a diameter of 10, 20, 30, 40, 50, 60, 70, 80, 90, or 100 mm from an injection site. In certain embodiments, the tissue is muscular tissue or heart tissue.

In certain embodiments, this disclosure relates to methods of enhancing blood vessel regeneration in the tissues where blood vessels have been damaged comprising administering a nucleic acid or vector encoding transcription factor ETV2 to a subject in a tissue wherein non-endothelial cells at the injection site convert into endothelial cells or vessels.

In certain embodiments, this disclosure relates to methods of blood vessel regeneration in a tissue. In certain embodiments, the blood vessels regeneration is within a diameter of 10, 20, 30, 40, 50, 60, 70, 80, 90, or 100 mm from an injection site. In certain embodiments, the tissue is muscular tissue or heart tissue.

In certain embodiments, the nucleic acid is mRNA encoding ETV2. In certain embodiments, the nucleic acid is DNA or RNA encoding ETV2. In certain embodiments, nucleic acid or vector encoding ETV2 is a recombinant lentivirus, retrovirus, adenovirus, or adeno-associated virus (AAV).

In certain embodiments, this disclosure relates to methods of enhancing blood vessel regeneration in the tissues where blood vessels have been damaged comprising administering an ETV2 protein or functional fragment thereof to a subject in a tissue wherein non-endothelial cells at the injection site convert into endothelial cells or vessels. In certain embodiments, the protein is contained within an exosome or other particulate structure.

In certain embodiments, this disclosure relates to methods of enhancing blood vessel regeneration in a damaged tissue. In certain embodiments, the damages blood vessels regeneration is within a diameter of 10, 20, 30, 40, 50, 60, 70, 80, 90, or 100 mm from an injection site. In certain embodiments, the tissue is muscular tissue or heart tissue.

In certain embodiments, this disclosure relates to methods of producing new blood vessels in tissues comprising administering a nucleic acid or vector encoding transcription factor ETV2 to a subject in a tissue wherein non-endothelial cells or endothelial cells at the injection site convert into blood vessels.

In certain embodiments, this disclosure relates to methods of producing new blood vessels in a tissue. In certain embodiments, the new blood vessels are within a diameter of 10, 20, 30, 40, 50, 60, 70, 80, 90, or 100 mm from an injection site. In certain embodiments, the tissue is muscular tissue or heart tissue.

In certain embodiments, the nucleic acid is mRNA encoding ETV2. In certain embodiments, the nucleic acid is DNA or RNA encoding ETV2. In certain embodiments, nucleic acid or vector encoding ETV2 is a recombinant lentivirus, retrovirus, adenovirus, or adeno-associated virus (AAV).

In certain embodiments, this disclosure relates to methods of producing new blood vessels in tissues comprising administering an ETV2 protein or functional fragment thereof to a subject in a tissue wherein non-endothelial cells or endothelial cells at the injection site convert into blood vessels. In certain embodiments, the protein is contained within an exosome or other particulate structure.

In certain embodiments, this disclosure relates to methods of treating disease requiring revascularization comprising administering a nucleic acid or vector encoding transcription factor ETV2 to a subject in a tissue wherein non-endothelial cells or endothelial cells at the injection site convert into blood vessels.

In certain embodiments, the disease requiring revascularization is coronary artery diseases, myocardial infarction, heart failure, peripheral artery diseases, critical limb ischemia, stroke, diabetic complications, and would healing. Other example diseases include angina, calcific aortic valve disease, hypertensive heart disease, rheumatic heart disease, cardiomyopathy, heart arrhythmia, congenital heart disease, valvular heart disease, carditis, aortic aneurysms, thromboembolic disease, and venous thrombosis.

In certain embodiments, the nucleic acid is mRNA encoding ETV2. In certain embodiments, the nucleic acid is DNA or RNA encoding ETV2. In certain embodiments, nucleic acid or vector encoding ETV2 is a recombinant lentivirus, retrovirus, adenovirus, or adeno-associated virus (AAV).

In certain embodiments, the tissues express increased levels of endothelium surface markers compared to the fibroblasts, wherein the surface markers are KDR, CDH5, PECAM1, TEK, or combinations thereof, thereby providing endothelial like cells.

In certain embodiments, use of a ETV2 pharmaceutical composition, protein, exosome, protein particle, nucleic acid or recombinant vector do or do not contain ERG or FLI1 protein or a nucleic acid that encodes ERG or FLI1 or do or do not contain FOXC2, MEF2C, SOX17, NANOG, or HEY1 protein or encoded nucleic acid.

In certain embodiments, this disclosure relates to methods of preventing or treating diseases requiring revascularization comprising administering an ETV2 protein or functional fragment thereof to a subject in a tissue wherein non-endothelial cells or endothelial cells at the injection site convert into blood vessels. In certain embodiments, the disease requiring revascularization is coronary artery diseases, myocardial infarction, heart failure, peripheral artery diseases, critical limb ischemia, stroke, diabetic complications, and would healing. In certain embodiments, the protein is contained within on the surface of an exosome or other particulate structure.

In certain embodiments, the cells within tissues express increased levels of endothelium surface markers compared to the normal tissue, wherein the surface markers are KDR, CDH5, PECAM1, TEK, or combinations thereof, thereby providing endothelial and/or vessels forming cells.

In certain embodiments, use of ETV2 materials do or do not contain ERG or FLI1 protein or a nucleic acid that encodes ERG or FIL1 or do or do not contain FOXC2, MEF2C, SOX17, NANOG, or HEY1 protein or nucleic acid.

### Pharmaceutical Compositions

ETV2 proteins, protein particles, exosomes, nucleic acids, recombinant expression vectors, host cells (including populations thereof), (collectively the ETV2 materials) can be formulated into a composition, such as a pharmaceutical composition. In this regard, this disclosure contemplates a pharmaceutical composition comprising any of the ETV2 proteins functional portions, functional variants, nucleic acids, expression vectors, host cells (including populations thereof), described herein, and a pharmaceutically acceptable carrier. This disclosure contemplates pharmaceutical compositions containing any of the ETV2 materials can comprise more than one polypeptide and a nucleic acid, or two or more different ETV2 materials. Alternatively, the pharmaceutical composition can comprise a ETV2 material in combination with another pharmaceutically active agent(s) or drug(s).

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil and viscoleo) and injectable organic esters such as ethyl oleate.

Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Preferably, the ETV2 material is administered by injection, e.g., intramuscularly or intramyocardially. The pharmaceutically acceptable carrier for injection may include any isotonic carrier such as, for example, normal saline (about 0.90% w/v of NaCl in water, about 300 mOsm/L NaCl in water, or about 9.0 g NaCl per liter of water), about 5% dextrose in water, or Ringer's lactate.

Providing a pharmaceutic composition is possible in a one-step process, simply by adding a suitable pharmaceutically acceptable diluent to the composition in a container. In certain embodiments, the container is preferably a syringe for administering the reconstituted pharmaceutical composition after contact with the diluent. In certain embodiments, the ETV2 materials can be filled into a syringe, and the syringe can then be closed with the stopper. A diluent is used in an amount to achieve the desired end-concentration. The pharmaceutical composition may contain other useful component, such as ions, buffers, excipients, stabilizers, etc.

A “dry” pharmaceutical composition typically has only a residual content of moisture, which may approximately correspond to the moisture content of comparable commercial products, for example, has about 12% moisture as a dry product. Usually, the dry pharmaceutical composition according to the present invention has a residual moisture content preferably below 10% moisture, more preferred below 5% moisture, especially below 1% moisture. The pharmaceutical composition can also have lower moisture content, e.g. 0.1% or even below. In certain embodiments, the pharmaceutical composition is provided in dry in order to prevent degradation and enable storage stability.

A container can be any container suitable for housing (and storing) pharmaceutically compositions such as syringes, vials, tubes, etc. The pharmaceutical composition may then preferably be applied via specific needles of the syringe or via suitable catheters. A typical diluent comprises water for injection, and NaCl (preferably 50 to 150 mM, especially 110 mM), CaCl2 (preferably 10 to 80 mM, especially 40 mM), sodium acetate (preferably 0 to 50 mM, especially 20 mM) and mannitol (preferably up to 10% w/w, especially 2% w/w). Preferably, the diluent can also include a buffer or buffer system so as to buffer the pH of the reconstituted dry composition, preferably at a pH of 6.2 to 7.5, especially at pH of 6.9 to 7.1.

In certain embodiments, the diluent is provided in a separate container. This can preferably be a syringe. The diluent in the syringe can then easily be applied to the container for reconstitution of the dry compositions. If the container is also a syringe, both syringes can be finished together in a pack. It is therefore preferred to provide the dry compositions in a syringe, which is finished with a diluent syringe with a pharmaceutically acceptable diluent for reconstituting, said dry and stable composition.

In certain embodiments, this disclosure contemplates a kit comprising a pharmaceutical composition disclosed herein and a container with a suitable diluent. Further components of the kit may be instructions for use, administration means, such as syringes, catheters, brushes, etc. (if the compositions are not already provided in the administration means) or other components necessary for use in medical (surgical) practice, such as substitute needles or catheters, extra vials or further wound cover means. In certain embodiments, the kit comprises a syringe housing the dry and stable hemostatic composition and a syringe containing the diluent (or provided to take up the diluent from another diluent container).

### Examples

Direct In Vivo Reprogramming of Fibroblasts into Endothelial Cells

The direct in vivo reprogramming of fibroblasts into endothelial cells was confirmed in a transgenic mouse strain (Fsp1-Cre;R26R-tdTomato) using two cardiovascular disease models, the hindlimb ischemia (HLI) model mimicking peripheral artery disease and the acute myocardial infarction (MI) model mimicking ischemic coronary artery disease. In this transgenic mouse strain, fibroblasts (FSP1+) express TdTomato red fluorescence protein and are labeled.

HLI was induced in mice aged at 12 weeks by removal of artery in hindlimb. In detail, mice were anesthetized with meloxicam-isoflurane. After removing hair on legs, artery exposure was obtained by performing an incision in the skin overlying the middle portion of a hindlimb and the femoral artery was dissected after ligating the proximal end of the femoral artery and the distal portion of the saphenous artery. Adenoviral ETV2 (1×10⁸ IFU/100 μl PBS/25 g mouse) was injected intramuscularly into the thigh muscle at 4-5 sites.

MI was induced in mice aged at 10 weeks by ligation of the left anterior descending coronary artery. In detail, mice were anesthetized with meloxicam-isoflurane. After mechanically removing hair on chest, betadine and alcohol disinfection was performed. Animals were located in the platform restraining with tape. Under mechanical ventilator assist, a 1-cm incision in the left parasternal 4th intercostal space was done. The heart was exposed and myocardial infarction (MI) was induced by ligating the left anterior descending coronary artery with 8-0 Prolene™ suture and the chest was evacuated following incision closure from ventilation. Incision was closed by continuous suturing by using non-absorbable sutures. Adenoviral ETV2 (5×10⁷ IFU/50 μl PBS/25 g mouse) was injected directly into MI heart. At 4 weeks after injection, the mice were perfused with FITC-conjugated BSL1 lectin to label functional ECs in blood vessels and harvested the hindlimb muscle and heart tissues. The collected tissues were examined by confocal microscopy. Some TdTomato+ cells (FSP1+, fibroblasts) were detected to be co-localized with FITC (BSL1-lectin), suggesting direct reprogramming of fibroblasts into functional ECs. A portion of TdTomato+ cells were incorporated into the vessels (BSL1-lectin), while some were not. These results confirmed that injection of Ad-ETV2 is able to induce reprogramming of in vivo fibroblasts into ECs, which can further contribute to vessel formation.

MI Induced Mice Injected with Ad-ETV2

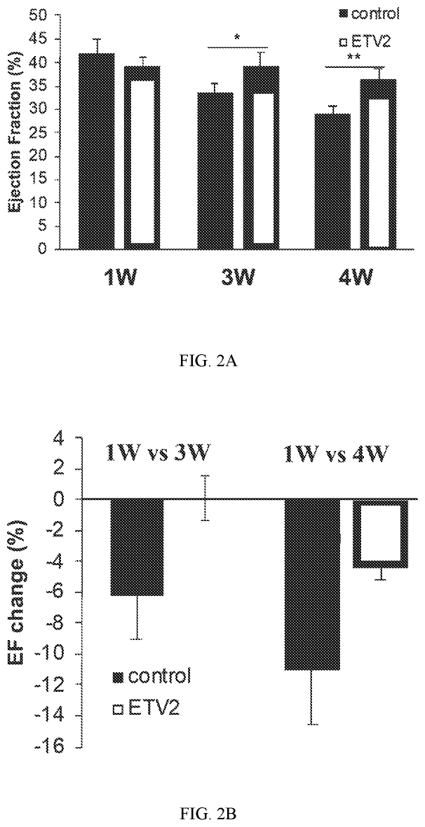

Echocardiography was performed on MI induced mice injected with Ad-ETV2 or control at week one, three and four after the surgery to assess the function of the hearts (FIG. 2A-B). Ad-ETV2 injected mice showed conserved ejection fraction during the progression of decrease by the passage of the time in control group (FIG. 2A). Fold changes between ejection fraction of week three and four verses week one showed that Ad-ETV2 injection blocked progression of decreased heart function (FIG. 2B). These data indicate cardiac function was improved by Ad-ETV2 injection.

### Mouse Models

The therapeutic effect of ETV2 in cardiovascular disease will be examined further in both MI and HLI model in mouse. At 1, 3, 4 wks, 2, 4 and 6 month post injection of ETV2, the therapeutic effect will be determined in following four criteria. For HLI, 1) improved recovery of ischemic injury by ETV2 delivery evaluated by the degree of the limb loss at 4 weeks post ischemic injury, 2) improved blood flow in the ischemic hindlimb evaluated by Laser Doppler Perfusion Imaging (LDPI) at D0, D3, D7, D14, D21, and D28 post ischemic injury, 3) the neovascularization effects assessed by microCT at 1, 2, 4 wks, 3, 6, and 10 month post surgery for the analysis of the overall vascularity in hindlimb: vessel number, volume, diameter, separation, connectivity, and remodeling of the vessels in the hindlimb ischemia model, and 4) the increase of capillary density assessed by immunohistochemistry of ischemic hindlimb muscle tissue with PECAM1 antibody or perfusion with BSL1. For MI, 1) the extent of cardiac fibrosis assessed by Masson's Trichrome staining, 2) the neovascularization effects assessed by microCT, and 3) the increase of capillary density assessed by immunohistochemistry of MI heart tissue. Adenoviral ETV2 (or Lentiviral ETV2, AAV-ETV2) will be injected directly into MI heart or into induced ischemic hindlimb immediately after surgery. It is desired that ETV2 delivery will improve the recovery and enhance neovascularization

What is claimed:

1. A method of converting non-endothelial cells into endothelial cells comprising administering a nucleic acid or vector encoding transcription factor ETV2 to a subject in a tissue wherein non-endothelial cells in the tissue convert into endothelial cells.

2. The method of claim 1 wherein the nucleic acid is DNA or RNA.

3. The method of claim 1 wherein the nucleic acid is mRNA.

4. The method of claim 1 wherein the nucleic acid or vector encoding ETV2 is a recombinant lentivirus, retrovirus, adenovirus, or adeno-associated virus (AAV).

5. A method of converting non-endothelial cells into endothelial cells comprising administering an ETV2 protein or functional fragment thereof to a subject in places of the body or tissue wherein non-endothelial cells in the tissue convert into endothelial cells.

6. The method of claim 5 wherein the protein is contained within an exosome or other particulate structure.

7. A method of generating blood vessels in a tissue comprising administering a nucleic acid or vector encoding transcription factor ETV2 to a subject in a tissue wherein non-endothelial cells in the tissue convert into endothelial cells and vessels.

8. The method of claim 7 wherein the nucleic acid is DNA or RNA.

9. The method of claim 7 wherein the nucleic acid is mRNA.

10. The method of claim 7 wherein the nucleic acid or vector encoding ETV2 is a recombinant lentivirus, retrovirus, adenovirus, or adeno-associated virus (AAV).

11. A method of generating blood vessels in a tissue comprising administering an ETV2 protein or functional fragment thereof to a subject in a tissue wherein non-endothelial cells in the tissue convert into endothelial cells and vessels.

12. The method of claim 11 wherein the protein is contained within an exosome or other particulate structure.

13. A method of enhancing blood vessel regeneration in the tissues where blood vessels have been damaged comprising administering a nucleic acid or vector encoding transcription factor ETV2 to a subject in a tissue wherein non-endothelial cells in the tissue convert into endothelial cells and vessels.

14. The method of claim 13 wherein the nucleic acid is DNA or RNA.

15. The method of claim 13 wherein the nucleic acid is mRNA.

16. The method of claim 13 wherein the nucleic acid or vector encoding ETV2 is a recombinant lentivirus, retrovirus, adenovirus, or adeno-associated virus (AAV).

17. A method of enhancing blood vessel regeneration in the tissues where blood vessels have been damaged comprising administering an ETV2 protein or functional fragment thereof to a subject in a tissue wherein non-endothelial cells in the tissue convert into endothelial cells and vessels.

18. The method of claim 17 wherein the protein is contained within an exosome or other particulate structure.

19. A method of producing new blood vessels in tissues comprising administering a nucleic acid or vector encoding transcription factor ETV2 to a subject in a tissue wherein non-endothelial cells in the tissue convert into endothelial cells and vessels.

20. The method of claim 19 wherein the nucleic acid is DNA or RNA.

21. The method of claim 19 wherein the nucleic acid is mRNA.

22. The method of claim 19 wherein the nucleic acid or vector encoding ETV2 is a recombinant lentivirus, retrovirus, adenovirus, or adeno-associated virus (AAV).

23. A method of producing new blood vessels in tissues comprising administering an ETV2 protein or functional fragment thereof to a subject in a tissue wherein non-endothelial cells in the tissue convert into endothelial cells and vessels.

24. The method of claim 23 wherein the protein is contained within an exosome or other particulate structure.

25. A method of treating disease requiring revascularization comprising administering a nucleic acid or vector encoding transcription factor ETV2 to a subject in a tissue wherein non-endothelial cells in the tissue convert into endothelial cells and vessels.

26. The method of claim 25 wherein the nucleic acid is DNA or RNA.

27. The method of claim 25 wherein the nucleic acid is mRNA.

28. The method of claim 25 wherein the nucleic acid or vector encoding ETV2 is a recombinant lentivirus, retrovirus, adenovirus, or adeno-associated virus (AAV).

29. The method of claim 25 wherein the disease requiring revascularization is coronary artery diseases, myocardial infarction, heart failure, peripheral artery diseases, critical limb ischemia, stroke, diabetic complications, and would healing.

30. A method of treating disease requiring revascularization comprising administering an ETV2 protein or functional fragment thereof to a subject in a tissue wherein non-endothelial cells in the tissue convert into endothelial cells and vessels.

31. The method of claim 30 wherein the protein is contained within an exosome or other particulate structure.

32. The method of claim 30 wherein the disease requiring revascularization is coronary artery diseases, myocardial infarction, heart failure, peripheral artery diseases, critical limb ischemia, stroke, diabetic complications, and would healing.
